(19) United States
(12) Patent Application Publication
(10) Pub. No.: US 2025/0131644 A1
(43) Pub. Date: Apr. 24, 2025

(54) QUANTIFICATION OF INTRAOCULAR DIMENSIONS AND VOLUMES

(71) Applicant: Vanderbilt University, Nashville, TN (US)

(72) Inventors: Yuankai Tao, Nashville, TN (US); Jacob Watson, Nashville, TN (US); Rachel Hecht, Nashville, TN (US); Alicia Repka, Nashville, TN (US)

(21) Appl. No.: 18/916,320

(22) Filed: Oct. 15, 2024

Related U.S. Application Data
(60) Provisional application No. 63/591,882, filed on Oct. 20, 2023.

(51) Int. Cl. G06T 15/08; G06T 7/00; G06T 7/10; G06T 7/62; G06T 15/06; G06V 10/25; G16H 20/17

(52) U.S. Cl. CPC G06T 15/08; G06T 7/0012; G06T 7/10; G06T 7/62; G06T 15/06; G06V 10/25; G16H 20/17; G06T 2207/30041; G06V 2201/07

(57) ABSTRACT

Methods and apparatus for quantifying an injected volume in an area of tissue during a medical procedure. In one example, a method includes determining a pixel-to-size scale parameter based on a pixelated image of a reference object included in a FOV of an optical instrument configured to perform volumetric imaging, the FOV further including an injection site; computing a first 3D model of the injected volume in the area of tissue based on a volumetric image of the FOV obtained using the optical instrument; refining the first 3D model to obtain a second 3D model of the injected volume, the refining including correcting a shape and a size of the first 3D model to reduce distortions associated with light refraction at a boundary of the injected volume in the area of tissue; and calculating a value of the injected volume based on the second 3D model and further based on the pixel-to-size scale parameter.

### CROSS-REFERENCE TO RELATED APPLICATIONS

This application is a non-provisional of and claims the benefit of U.S. Provisional Patent Application No. 63/591,882 filed on Oct. 20, 2023, and entitled “SYSTEMS AND METHODS FOR QUANTIFICATION OF INTRAOCULAR DIMENSIONS AND VOLUMES,” the contents of which are incorporated herein by reference.

### STATEMENT REGARDING FEDERALLY SPONSORED RESEARCH

This invention was made with government support under EY030490, EY031769, and EY033969 awarded by the National Institutes of Health. The government has certain rights in the invention.

### FIELD OF THE DISCLOSURE

Various example embodiments relate to optical instruments and, more specifically but not exclusively, to surgical microscopes and associated methods.

### BACKGROUND

An operating or surgical microscope is an optical microscope specifically designed for use in a surgical setting, usually to assist with microsurgery. Typical magnification provided by a surgical microscope is in the approximate range from 4× to 40×. Fields of medicine that make significant use of surgical microscopes include plastic surgery, dentistry (e.g., endodontics), otolaryngology (or ENT) surgery, ophthalmic surgery, and neurosurgery.

### SUMMARY

Some examples provide improved methods and apparatus for volumetric estimation of subretinal injection volumes during ophthalmic surgery based on intraocular reference features tracked with intraoperative optical coherence tomography. In one example, a surgical instrument, such as the ophthalmic forceps or endo-illuminator, is used as a reference object in the field of view (FOV). In some examples, a customized object-detection neural network is used to constrain mask outputs of the Segment Anything Model. Spline interpolation is used to combine frame segmentations into a single three-dimensional (3D) volume. Physical volumes are then estimated by implementing more precise distortion and 3D-refraction corrections and by scaling the segmented volumes by a voxel-to-microliter conversion parameter. In at least some use cases, the provided improvements will benefit real-time ophthalmic surgical decision-making, e.g., by enabling more precise subretinal microliter volume injections for drug delivery and gene therapy.

In one example, a medical system comprises: a drug delivery system configurable to controllably inject a fluid into an area of tissue of a patient; an optical instrument configured to perform volumetric imaging in a FOV including a surgical instrument and an injection site in the area of tissue; and an electronic controller configured to: determine a pixel-to-size scale parameter based on a pixelated image of the surgical instrument in the FOV; and estimate a volume of the fluid injected by the drug delivery system into the area of tissue based on a pixelated volumetric image of the FOV and further based on the pixel-to-size scale parameter.

In another example, a method of quantifying an injected volume in an area of tissue during a medical procedure comprises the steps of: determining a pixel-to-size scale parameter based on a pixelated image of a reference object included in a FOV of an optical instrument configured to perform volumetric imaging, the FOV further including an injection site in the area of tissue; computing a first 3D model of the injected volume in the area of tissue based on a volumetric image of the FOV obtained using the optical instrument; refining the first 3D model to obtain a second 3D model of the injected volume in the area of tissue, the refining including correcting a shape and a size of the first 3D model to reduce distortions associated with light refraction at a boundary of the injected volume in the area of tissue; and calculating a value of the injected volume based on the second 3D model and further based on the pixel-to-size scale parameter.

According to yet another example embodiment, provided is a non-transitory computer-readable medium storing instructions that, when executed by an electronic processor, cause the electronic processor to perform operations comprising the above method.

### BRIEF DESCRIPTION OF THE DRAWINGS

Other aspects, features, and benefits of various disclosed embodiments will become more fully apparent, by way of example, from the following detailed description and the accompanying drawings, in which:

FIG. 1 is a block diagram illustrating a medical system in which at least some embodiments can be practiced according to some examples.

FIG. 2 is a block diagram illustrating an optical microscope used in the medical system of FIG. 1 according to various examples.

FIGS. 3A-3B are block diagrams illustrating a volumetric imaging module coupled to the optical microscope of FIG. 2 according to some examples.

FIG. 4 is a flowchart illustrating a method of quantifying intraocular dimensions and volumes implemented in the medical system of FIG. 1 according to various examples.

FIGS. 5A-5F graphically illustrate a time series of 3D models generated with the method of FIG. 4 according to one example.

FIGS. 6A-6D pictorially illustrate images including a surgical instrument used for pixel size referencing in the method of FIG. 4 according to one example.

FIG. 7 is a block diagram illustrating a computing device used in or connected to the medical system of FIG. 1 according to some examples.

### DETAILED DESCRIPTION

In the following description, numerous details are set forth, such as optical device configurations, timings, operations, and the like, in order to provide an understanding of one or more aspects of the present disclosure. It will be readily apparent to one skilled in the art that these specific details are mere examples and are not intended to limit the scope of this application.

In some examples, a surgical microscope may incorporate a beamsplitter that allows splitting of the pertinent light beam to enable the surgeon's assistant to also visualize the procedure or to allow photography or videography of the surgical field to be performed substantially without any interference with the surgical procedure. In some examples, a surgical microscope may incorporate optics that enables intraoperative optical coherence tomography (iOCT) to be performed during the procedure. Microscope-integrated iOCT can beneficially be used, e.g., for depth-resolved volumetric imaging during surgery. In some examples, iOCT provides depth-resolved visualization of retinal microstructures and dynamics during ophthalmic surgery. In some examples, real-time feedback to the surgeon can be provided based on three-dimensional (3D) iOCT imaging with image-based quantitative metrics, such as tissue deformation and intraocular volume measurements, as a means to improving surgical outcomes and supporting a wider array of surgical maneuvers.

As used herein, the term “real time” refers to a computer-based process that controls or monitors a corresponding environment by receiving data, processing the received data, and generating a response sufficiently quickly to affect or characterize the environment without significant delay. In the context of control or processing software, real-time responses are often understood to be on the order of milliseconds, or sometimes microseconds. In the context of a surgical procedure, “real-time” updates mean that the experimental data and measurement results derived therefrom sufficiently accurately represent the state of the surgical field of view (FOV) at any point in time. In this case, data-acquisition and/or processing delays of several seconds may still be considered to be within “real time” or “near real time” for at least some surgical procedures.

Conventionally, during subretinal deliveries of drug and gene therapies in the course of ophthalmic surgery, surgeons rely on visual estimations, infusion durations, or microinjector readings to approximately determine and track the injection volumes. However, such reliance often disadvantageously leads to relatively high variability of the actual delivered volumes of the drug. As such, at least some of the iOCT imaging and segmentation approaches disclosed herein below are directed toward beneficially achieving more-accurate quantification of subretinal injection volumes in both in vivo surgical procedures and ex vivo validation studies.

In various examples, the disclosed systems and methods may implement one or more of the following features:
Quantification of spatial dimensions using a known reference feature, which may include:
   Utilizing a moving reference feature within an unknown field to quantify spatial dimensions;
   Utilizing a stationary reference feature within an unknown field to quantify ranging in-depth; and/or
   Providing estimations of feature and field scaling across varying points within a reference field.
Quantification of anatomical features of unknown spatial dimensions using a known reference feature, which may include:
   Quantifying ocular anatomy in both posterior and anterior fields; and/or
   Quantifying topographic deformations of anatomical features.
Correction in non-uniform fields based on a known reference feature, which may include:
   Use of ray casting through known optics, known index of refraction, and curvature of optical surfaces to correct for distortions in non-uniform fields and non-uniform scaling across fields.
Use of live imaging to quantify information on spatial unknown fields or corrected non-uniform fields, which may include:
   Quantifying selected non-uniform features, such as distortion, parallax, and feature changes based on the distance to a target surface.
Quantification of temporal metrics within an unknown field, which may include:
   Quantifying temporal information using a known internal reference feature.
Use of a known internal-reference dimension to aid in image mosaicking within uniform and non-uniform fields.
Real-time, closed-loop feedback of volumetric injections based on acquired segmentation data.
Quantification of an unknown spatial field using a known reference feature from one or more other imaging modalities (e.g., microscopy, photography, endoscopy, ultrasound, etc.).

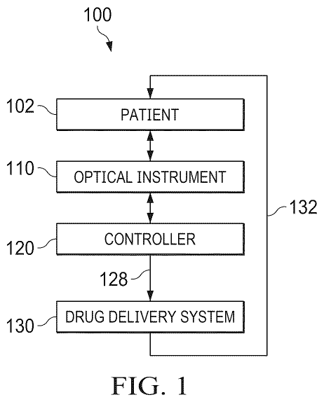

FIG. 1 is a block diagram illustrating a medical system 100 in which at least some embodiments can be practiced according to various examples. The medical system 100 includes an automated drug delivery system 130 configured to controllably inject a fluid 132 containing a drug or therapeutic substance into a selected organ or area of tissue of a patient 102. In a representative example, the drug delivery system 130 includes a reservoir, a pump, and an injecting device (not explicitly shown). The reservoir is filled with a relatively large fluid volume of the drug or therapeutic substance 132. The pump is connected between the reservoir and the injecting device and operates to draw a specified amount (e.g., volume) of the fluid 132 from the reservoir and transfer it into the injecting device. The injecting device typically includes a needle, the tip of which is properly positioned by the operating surgeon in or at the selected organ of the patient 102. The volume of the fluid 132 drawn and transferred by the pump exits the tip of the needle, thereby being delivered to the organ of the patient 102. In some examples, the organ into which the fluid 132 is injected is a human eye. In other examples, other organs can similarly be subjected to drug or therapeutic substance injections.

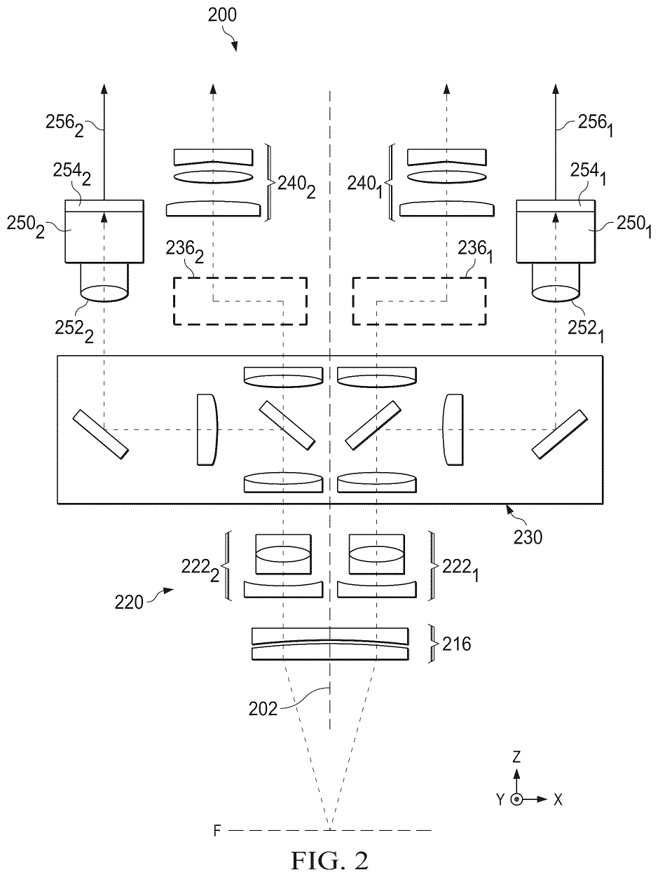

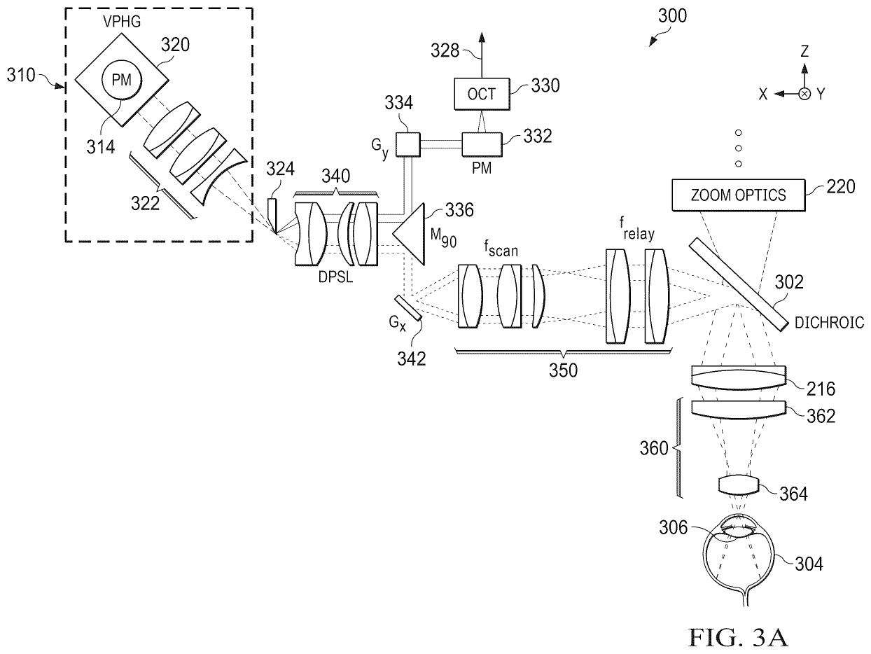

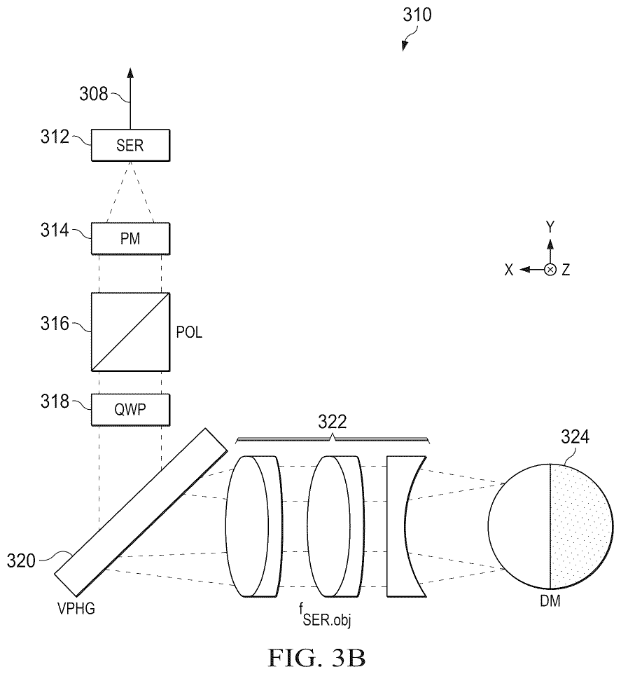

The medical system 100 also includes an optical instrument 110 and an electronic controller 120. The optical instrument 110 is optically coupled to the organ of the patient 102 into which the fluid 132 is being injected such that optical measurements can be carried out, based on which the volume of the injected fluid 132 can be quantified. Representative examples of the optical instrument 110 are described in more detail below in reference to FIGS. 2, 3A, and 3B.

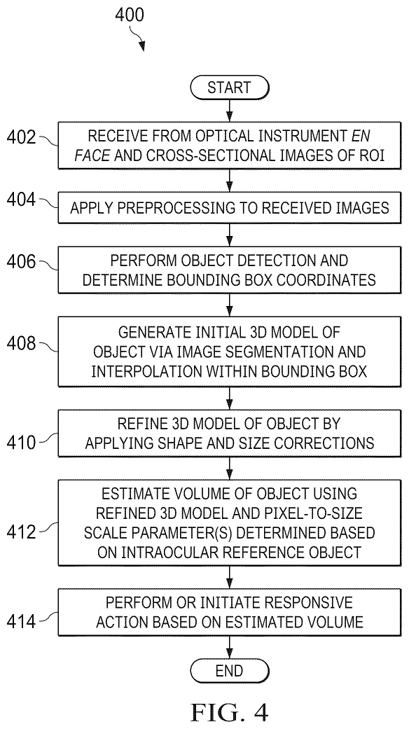

The electronic controller 120 is configured to perform processing of the experimental data acquired with the optical instrument 110 to accurately estimate the injected volume of the fluid 132. Examples of data and image processing performed by the electronic controller 120 for this purpose are described in more detail below in reference to FIGS. 4-6. Based on the obtained volume estimate, the electronic controller 120 operates to generate a corresponding control signal 128 for the drug delivery system 130. In various examples, in response to the control signal 128, the drug delivery system 130 may operate the pump and/or other pertinent components thereof to inject a specified volume of the fluid 132 into the organ of the patient 102, regulate the flow rate of the fluid 132 into the patient, or stop the delivery of the fluid 132. Example drug or therapeutic substance delivery methods implemented in the medical system 100 may be based on the method 400 described in more detail below in reference to FIGS. 4-6.

FIG. 2 is a block diagram illustrating an optical microscope 200 used in the optical instrument 110 of the medical system 100 according to various examples. In some examples, the optical microscope 200 can be configured to image an eye retina of the patient 102 (e.g., see FIG. 3A). In such examples, the optical microscope 200 is additionally outfitted with binocular indirect ophthalmo-microscope (BIOM) optics (not explicitly shown in FIG. 2; see the element 360 in FIG. 3A) positioned between an objective lens 216 and the eye. In other examples, the optical microscope 200 can be configured to image other organs of the patient 102.

The optical microscope 200 includes first and second eyepieces 240₁ and 240₂. The first eyepiece 240₁ is optically coupled to the objective lens 216 via a first portion 2221 of a magnification changer (zoom optics) 220 as indicated in FIG. 2. The second eyepiece 240₂ may be nominally identical to the first eyepiece 240₁ and is similarly optically coupled to the objective lens 216 via a second portion 2222 of the magnification changer 220. The first and second eyepieces 240₁ and 240₂ are typically arranged in a binocular head of the optical microscope 200 with their optical axes being substantially parallel to one another and spatially separated by a distance corresponding to the interpupillary distance of the user. In some examples, the binocular head of the optical microscope 200 enables the distance between the first and second eyepieces 240₁ and 240₂ to be adjustable to compensate for interpupillary distance variations in different users and/or uneven vision.

Different configurations of the optical microscope 200 may employ different embodiments of the eyepiece 240; (where i=1, 2) characterized by different respective magnifications, such as 10×, 12.5×, 16×, 20×, etc. The choice of magnification typically depends on the selected size of the FOV and the desired overall magnification of the optical microscope 200. In some examples, the eyepiece 240; has a focal length of 125 mm.

The magnification changer 220 is designed to change the degree of magnification of the optical microscope 200 without any change in the working distance (which is exemplified in FIG. 2 by the distance between the objective lens 216 and a focal plane F). In the example shown, the structure of the magnification changer 220 is symmetric with respect to the symmetry plane passing through an optical axis 202 of the microscope 200 and incorporates a system of lenses, relative position(s) of which can be controllably changed to provide a continuous change in the magnification. In one example, the changeable magnification provided by the magnification changer 220 can be in the range from 0.5× to 2.5×.

The optical microscope 200 also includes a camera adapter 230 designed and configured to enable simultaneous observation of the microscope's FOV by the user through the first and second eyepieces 240₁ and 240₂ and the corresponding image capture by one or both of cameras 250₁, 250₂. In the example shown, the camera 250ᵢ (where i=1, 2) includes a camera lens 252ᵢ and a pixelated photodetector (e.g., a CCD) 254ᵢ, which are optically coupled to the objective lens 216 through the camera adapter 230 and the corresponding portion 222ᵢ of the magnification changer 220 as indicated in FIG. 2. The captured images can be read out from the pixelated photodetector 254ᵢ in a conventional manner via a respective readout signal 256ᵢ, which may then be routed to the electronic controller 120 (FIG. 1) for data processing and/or storage.

The optical microscope 200 also includes inverting prisms 236₁, 236₂ positioned between the camera adapter 230 and the eyepieces 240₁, 240₂ as indicated in FIG. 2. In operation, the inverting prisms 236₁, 236₂ perform correction for the inverted image formed by the eyepieces 240₁, 240₂. In some examples, the inverting prisms 236₁, 236₂ are implemented using a pair of Porro-Abbe prisms. In other examples, other suitable prism implementations may also be used.

The illumination light for the optical microscope 200 is typically generated by an external illuminator (not explicitly shown in FIG. 2), e.g., a suitable visible light source that is installed away from the optical microscope 200 to avoid undesired heating of the microscope optics and/or of the surgical site. In various examples, the external illuminator may include a xenon light bulb, a halogen light bulb, an LED source, etc. In some examples, the light generated by the external illuminator is transmitted to the optical microscope 200 through a fiber guide and then passes through the objective lens 216 to illuminate the FOV. The illumination-light intensity can be varied by changing the voltage(s) applied to the light bulb(s) or LED source(s). While various designs of external illuminators are available, a preferred design for ophthalmic surgery provides for coaxial illumination. The coaxial illumination beneficially allows the illumination light to follow the same path as the object light to avoid shadows, which might occur with oblique illumination in some cases. In some examples, a light pipe or endo-illuminator can also be used to selectively illuminate a portion of the FOV.

In various examples, the optical microscope 200 may use one of the following mechanical support systems: (i) on casters; (ii) wall mounted; (iii) tabletop; and (iv) ceiling mounted. In some cases, an on-caster stand is the preferred mechanical support structure owing to its enhanced mobility. In some other cases, a ceiling or wall mount may be preferred because it helps with space management. An example mechanical support system for the optical microscope 200 may include precision motorized mechanics so that the microscope can be adjusted flexibly to the right position as needed. In some examples, the mechanical support system incorporates a foot pedal that can be used to control the illumination, focus, zoom, and X-Y position of the optics over the surgical field.

FIGS. 3A-3B are block diagrams illustrating a volumetric imaging module 300 coupled to the optical microscope 200 according to some examples. More specifically, FIG. 3A schematically illustrates a side view of the volumetric imaging module 300. FIG. 3B schematically illustrates a bottom view of a spectrally encoded reflectometry (SER) sub-module 310 of the volumetric imaging module 300. Note that the XYZ coordinate triad shown in FIGS. 3A-3B has the same orientation as the XYZ coordinate triad shown in FIG. 2. In operation, the volumetric imaging module 300 enables both en face and vertical cross-section imaging of the corresponding FOV. Herein, the term “cross-section imaging” refers to the cross section along the spatial dimension orthogonal to the en face FOV of the optical microscope 200. With the shown XYZ coordinate triad, an en face imaging plane is parallel to the XY coordinate plane, and a complementary vertical “cross-sectional” dimension is parallel to the Z coordinate axis. The “top” direction is along the Z coordinate axis toward the zoom optics 220, and the “bottom” direction is along the Z coordinate axis toward an eye 304 (see FIG. 3A).

The volumetric imaging module 300 is designed and configured to perform spectrally encoded coherence tomography and reflectometry (SECTR), which combines cross-sectional swept-source optical-coherence-tomography (OCT) imaging with en face SER. This multimodality of the module 300 beneficially enables concurrent acquisition of en face reflectance images of region-of-interest (ROI) motion at high-speed with inherently spatiotemporally co-registered volumetric OCT data. The utility of the SECTR methodology was previously demonstrated, e.g., for SER-based retinal-tracking and OCT motion-correction, multi-volumetric OCT mosaicking to extend the imaging FOV, multi-volumetric averaging to improve the OCT signal-to-noise ratio (SNR) and OCT angiography connectivity.

Referring to FIG. 3A, the volumetric imaging module 300 is coupled to the imaging optics of the optical microscope 200 using a dichroic filter (or mirror) 302. In the example shown, the dichroic filter 302 is placed between the objective lens 216 and the magnification changer (zoom optics) 220 of the optical microscope 200 (also see FIG. 2). In one implementation, the dichroic filter 302 is substantially transparent to visible light and is highly (e.g., >90%) reflective to near infrared (NIR) light.

The volumetric imaging module 300 is configured to use NIR light generated by an external optical engine (not explicitly shown in FIG. 3A) to scan the FOV. In one example, the external optical engine includes a 400 kHz bidirectional 1051±46 nm swept laser. The optical output of this laser is power split approximately evenly into two optical portions, which are then directed to the SER sub-module 310 and an OCT sub-module 330, respectively, of the module 300. Example optical engines that can be used with the sub-modules 310 and 330 are described in more detail, e.g., in (1) Mohamed T. El-Haddad, Ivan Bozic, and Yuankai K. Tao, “Spectrally Encoded Coherence Tomography and Reflectometry (SECTR): simultaneous en face and cross-sectional imaging at 2 gigapixels-per-second,” J. Biophotonics, April 2018, Vol. 11 (4): e201700268, 21 pages; and (2) Jacob J. Watson, Rachel Hecht, and Yuankai K. Tao, “Optimization of handheld spectrally encoded coherence tomography and reflectometry for point-of-care ophthalmic diagnostic imaging,” Journal of Biomedical Optics, July 2024, Vol. 29 (7), 076006-(17 pages), both of which are incorporated herein by reference in their entirety.

Referring to both FIGS. 3A and 3B, the SER sub-module 310 includes a SER input/output block 312, a parabolic mirror (PM) 314, a linear polarizer (POL) 316, a quarter-wave plate (QWP) 318, a volumetric phase holographic grating (VPHG) 320, and a SER objective lens 322. SER illumination is collimated using the parabolic mirror 314 to maximize optical throughput. The linear polarizer 316 and the quarter wave plate 318 are used to implement circularly polarized SER illumination and cross-polarization detection. The optical beam impinging onto the VPHG 320 is spectrally dispersed thereby, and the resulting spectrally dispersed beam is focused by the telecentric SER objective lens 322 into a spectrally encoded line at the flat edge of a D-shaped pickoff mirror (DM) 324.

Referring to FIG. 3A, OCT illumination is collimated using an off-axis parabolic mirror (PM) 332 and then scanned by a slow-axis galvanometer (G_{y}) 334, reflected by a 90-degree prism mirror (M₉₀) 336, and focused at the DM 324 using a telecentric double-pass scan lens (DPSL) 340. The OCT and SER focused lines are combined across the DM 324 with minimal lateral offset to ensure overlapping SER and OCT FOVs. The downstream SER/OCT shared optics includes the DPSL 340, a fast-axis galvanometer (Gₓ) 342, a 5× magnifying 4F relay (f_{scan} to f_{relay}) 350, the dichroic filter 302, and a portion of the microscope optics including the objective lens 216. In the example shown, the downstream optics also includes the above-mentioned BIOM optics 360, which includes a reduction lens 362 and an ophthalmic lens 364. The BIOM optics 360 operates to reduce the beam dimeter and collimate the illumination light entering the patient eye 304 through a crystalline lens 306 thereof and further operates to properly couple the reflected light exiting the patient eye 304 through the crystalline lens 306 into the objective lens 216. When the optical microscope 200 is used to image objects other than the patient eye 304, the BIOM optics 360 may be absent. The 5× magnification of the relay 350 is used to compensate for the demagnification of the BIOM optics 360 during posterior eye imaging.

The returned SER signal is detected in the SER input/output block 312 using an avalanche photodiode (APD, not explicitly shown). The electrical signal generated by the APD is converted into digital form using an analog-to-digital converter (ADC, not explicitly shown), and a resulting digital signal 308 (see FIG. 3B) is then directed to the electronic controller 120 for processing. The returned OCT signal is combined with the corresponding optical reference signal in the OCT sub-module 330, and the resulting combined optical signal is detected using a balanced photodetector (BPD, not explicitly shown). The electrical signal generated by the BPD is converted into digital form using an ADC (not explicitly shown). A resulting digital signal 328 (see FIG. 3A) is then directed to the electronic controller 120 for processing.

FIG. 4 is a flowchart illustrating a method 400 of quantifying intraocular volumes implemented in the medical system 100 according to various examples. In some examples, the method 400 is executed using a program code running on a processor of the electronic controller 120. An example input to such program code includes a sequence of co-registered SER and OCT images of the ROI acquired using the optical microscope 200 (FIG. 2) equipped with the volumetric imaging module 300 (FIGS. 3A-3B). An example output of the program code includes an estimated physical-volume value of the selected observed intraocular structure or object, such as a bleb or an injected fluid volume. For illustration purposes and without any implied limitations, some features of the method 400 are described below with specific reference to ophthalmic surgery. From the provided description, a person of ordinary skill in the pertinent art will readily understand how to adapt the method 400 to other use cases, without any undue experimentation.

In a block 402 of the method 400, the electronic controller 120 receives from the optical instrument 110 a set of images of the ROI. In a typical example, the received images have been acquired using the optical microscope 200 (FIG. 2) equipped with the volumetric imaging module 300. In one example, the volumetric imaging module 300 is configured to use a 400-kHz 1060-nm swept-source with 1.4 mW OCT power and 5.8 mW 40-degree extended-source SER power. SER images are sampled at 2048×1000 (spectral×lateral) samples for a frame rate of 400 Hz. OCT volumes are concurrently sampled at 2048×1000×1000 (spectral×lateral×frames) samples for a volume acquisition time of approximately 2.5 seconds.

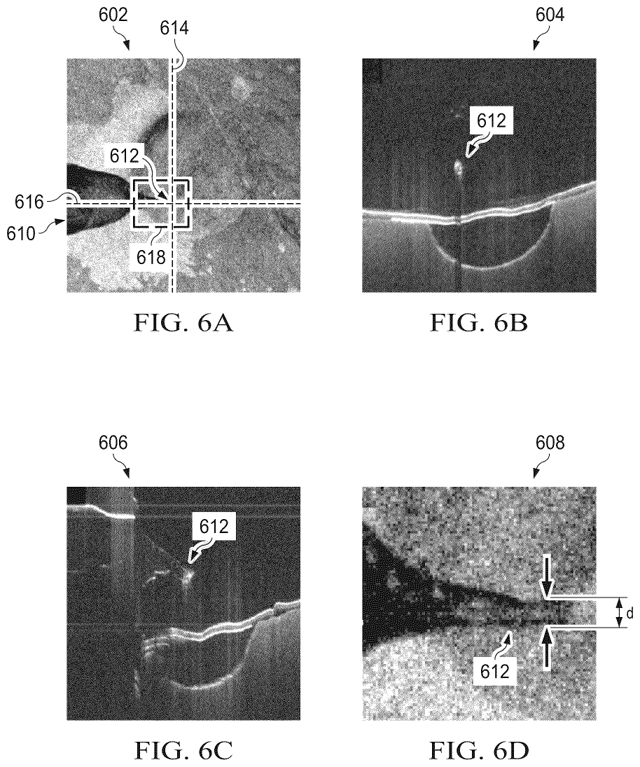

In a block 404 of the method 400, the electronic controller 120 operates to apply a set of one or more preprocessing operations to the image frames received in the block 402. In various examples, the preprocessing operations performed in the block 404 are configured to decrease the dataset size, increase contrast, and/or reduce shadowing artifacts introduced by the surgical instrument or intraocular reference object typically present in the images (e.g., see FIG. 6A). In some examples, the set of preprocessing operations performed in the block 404 may include operations selected from the group consisting of downsampling, averaging, cropping, thresholding, rescaling, shadow identification, and shadow-artifact reduction by interpolation. In one example, the OCT volume frames are down-sampled by a factor of four in the axial and fast-axis scanning dimensions and cropped to a ROI surrounding the object of interest. Corresponding frames across five sequential OCT volumes are averaged to reduce speckle noise while maintaining a clear delineation of object's edges. Any shadowing artifacts present in the ROI are filled in using a suitable computationally efficient interpolation method.

In a block 406 of the method 400, the electronic controller 120 operates to perform object detection in the preprocessed images generated in the block 404. In some examples, operations of the block 406 include the electronic controller 120 concatenating several individual frames into a corresponding single concatenated image. One purpose of this concatenation is to increase computational efficiency of the subsequent object-detection operations performed in the block 406 and/or of image-segmentation operations performed in the next block (labeled 408) of the method 400.

The electronic controller 120 is further configured to employ, in the block 406, a suitable object-detection algorithm to detect an object of interest in the concatenated images representing slices of the volumetric image. In the context of ophthalmic surgery, the object of interest may be a bleb or an injected fluid volume. In other use cases, other objects of interest can similarly be detected. In general, object detection deals with localizing a ROI within a bigger image and classifying the ROI in a manner similar to that of a typical image classifier. In some examples, a single image may include several regions of interest corresponding to different objects or classes. Object detection algorithms can broadly be divided into two categories based on how many times the same input image is passed through the corresponding neural network. Single-shot object detection uses a single pass of the input image to make predictions about the presence and location of one or more objects in the image. As such, single-shot object-detection algorithms process the entire image in a single pass, making these algorithms relatively computationally efficient. Two-shot object detection uses two passes of the input image to make predictions about the presence and location of one or more objects. The first pass is used to generate a set of proposals or potential object locations, and the second pass is used to refine these proposals and make “final” predictions.

In some examples, the object-detection neural network employed in the block 406 of the method 400 is implemented using a “You Only Look Once” (YOLO) neural network that makes predictions of bounding boxes and class probabilities. The YOLO model is a single-shot object detector that is based on a fully convolutional neural network (CNN). In some examples, the YOLO model takes an image as an input and then uses a deep CNN to detect objects in the image. The first 20 convolution layers of the model are pre-trained using ImageNet by plugging in a temporary average pooling and fully connected layer. Then, this pre-trained model is adapted to perform more-customized object detection by adding convolution and connected layers to improve performance. Once trained and connected, the YOLO's final fully connected layer predicts both class probabilities and bounding box coordinates. In one example, the YOLO v7 or v8 version may be used in the block 406.

In a block 408 of the method 400, the electronic controller 120 is configured to generate an approximate 3D model of the object of interest. In some examples, operations of the block 408 include image segmentation and object-boundary definition and smoothing. The image segmentation is constrained to the areas within the object bounding box(es) determined in the block 406. The object-boundary definition includes combining object segments corresponding to the pertinent class (e.g., bleb or drug injection) from a plurality of frames into an aggregated 3D segmentation volume and then constructing a 3D surface representing the boundary of this volume using smooth bivariate spline interpolation. The aggregated 3D segmentation volume having the boundary defined in this manner provides the approximate 3D model of the object of interest. This approximate 3D model is subjected to further refinement in the downstream blocks of the method 400, e.g., as described in more detail below.

In general, image segmentation is a process of dividing an image into multiple parts or regions that belong to the same class. In the context of ophthalmic surgery, image-segmentation operations of the block 408 are directed at identifying pixels belonging to the bleb or drug-injection class. In other use cases, other pertinent classes can also be used to configure the image-segmentation operations of the block 408. In various examples, different suitable image segmentation algorithms can be used in the block 408. For a specific use case, a corresponding image segmentation algorithm may be selected from the group consisting of a thresholding algorithm, a region growing algorithm, an edge-based segmentation algorithm, a clustering algorithm, a morphological-based segmentation algorithm, a watershed segmentation algorithm, an active contours algorithm, a Bayesian-based segmentation algorithm, a deep learning-based segmentation algorithm, a graph-based segmentation algorithm, and a superpixel-based segmentation algorithm. In some examples, the image-segmentation operations of the block 408 are implemented using a U-Net, which is a neural network having a U-shaped topology described, e.g., in Ronneberger, O., Fischer, P., and Brox, T., “U-net: Convolutional networks for biomedical image segmentation,” Medical Image Computing and Computer-Assisted Intervention-MICCAI 2015: 18th International Conference, Munich, Germany, Oct. 5-9, 2015, Proceedings, Part III 18, pp. 234-241. In some other examples, the image-segmentation operations of the block 408 are implemented using the Segment Anything Model (SAM) described, e.g., in Alexander Kirillov, Eric Mintun, Nikhila Ravi, et al., “Segment Anything,” arXiv:2304.02643. Both of the aforementioned publications are incorporated herein by reference in their entirety.

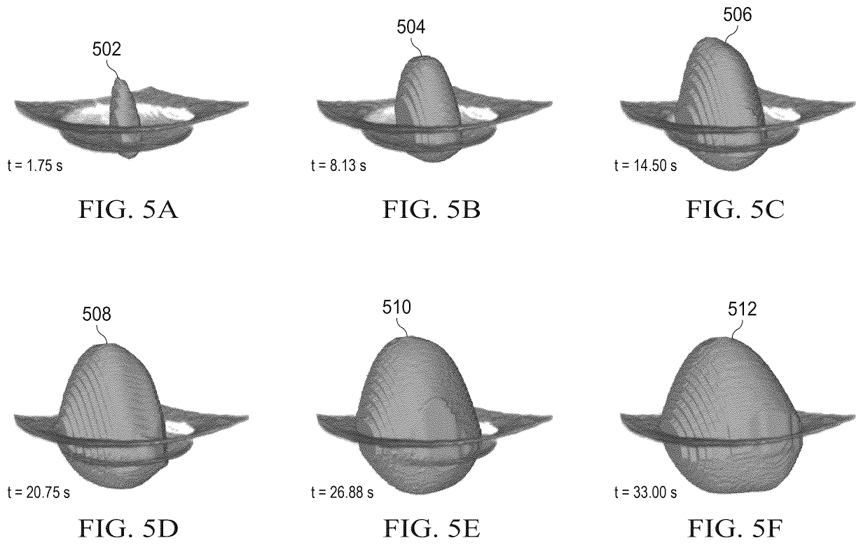

FIGS. 5A-5F graphically illustrate a time series of 3D models 502-512 generated in the block 408 of the method 400 according to one example. The 3D models 502-512 represent an experiment in which a subretinal injection was simulated by delivering a 5% w/w milk-water solution through a polyamide cannula with an outer diameter of 164 μm into a 3D-printed 25-mm axial length model eye. The solution was delivered into an elliptical well in the model eye designed to prevent lateral spreading of the resulting injected fluid volumes. The total duration of the injection was 33 seconds. The total volume of the fluid injection at 33 seconds was approximately 130 μL, and the corresponding approximate 3D model 512 is shown in FIG. 5F. The earliest approximate 3D model 502 corresponds to the time t=1.75 s after the start of the injection and is shown in FIG. 5A. The timestamps corresponding to the approximate 3D models 504, 506, 508, and 510 (FIGS. 5B-5E) are 8.13 s, 14.50 s, 20.75 s, and 26.88 s, respectively, from the start of the injection. Changes in the bleb size and shape in the course of the injection are readily observable in FIGS. 5A-5F.

Referring back to FIG. 4, in a block 410 of the method 400, the electronic controller 120 is configured to apply shape and size corrections to the approximate 3D model(s) generated in the block 408. In various examples, the shape/size corrections performed in the block 410 are directed at compensating or significantly reducing the distortions that are present in the approximate 3D model(s) generated in the block 408 due to: (i) the index of refraction contrast between the injected fluid volume and the eye's vitreous body, (ii) the generally complex curved shape of the injected fluid volume surface, and (iii) radial distortions across the retinal FOV. These factors typically cause the perceived (by the pixelated detectors of the optical instrument 110) shape and size of the injected fluid volume to be different form the actual physical shape and size. In one example, the shape and size correction operations of the block 410 include: (i) obtaining a 3D surface fit to the boundary of the approximate 3D model generated in the block 408; (ii) optical ray-casting through a top portion of the obtained 3D surface based on Snell's law; (iii) correcting a shape of the bottom portion of the obtained 3D surface based on the ray-casting; (iv) scaling the size of the 3D model based on the refractive index of the injected fluid volume and further based on the refractive index of tissue surrounding the injected fluid volume in the organ; and (v) corrections for the radial distortions. Application of the shape and size corrections to the approximate 3D model in the block 410 produces the corresponding adjusted and refined 3D model.

In a block 412 of the method 400, the electronic controller 120 is configured to determine an estimated value of the injected fluid volume based on the adjusted 3D model produced in the block 410. Note that the linear dimensions in the adjusted 3D model are measured in pixels, because the pixel is the natural granularity unit for the various images captured by the optical instrument 110. As such, the pixel units need to be converted into the corresponding physical size units (such as millimeters or micrometers) for the physical volume calculation.

To enable the determination of pixel-to-size scale parameters, the acquired images received by the electronic controller 120 in the block 402 of the method 400 typically include an intraocular reference object of known dimensions positioned within the FOV when the images are acquired by the optical instrument 110. In some examples, the reference object is a surgical instrument selected from the group consisting of ophthalmic forceps, an ophthalmic knife or blade, a light pipe, an endo-illuminator, a cannula, a needle, a surgical pick, a membrane brush, and a surgical scraper. In some examples, the time-dependent position of the surgical instrument within the FOV is automatically tracked in the sequence of frames using the tracking method described in U.S. Pat. No. 12,029,501, which is incorporated herein by reference in its entirety. Such tracking beneficially enables continuous scale referencing throughout the entire image sequence.

FIGS. 6A-6D pictorially illustrate images 602-608 used for pixel-to-size referencing in the block 412 of the method 400 according to one example. In the example shown, the images 602-608 include a subretinal cannula 610 having a tip 612. The image 602 (FIG. 6A) is an en face image. The image 608 (FIG. 6D) is a magnified view of the tip 612 corresponding to a box 618 indicated in the image 602. Marked as “d” in the image 608 is the known diameter of the tip 612 used as an internal reference dimension to measure the pixel-to-μm scale parameter. The image 604 (FIG. 6B) is an OTC image corresponding to a vertical cross-section plane 614 indicated in the image 602. The image 606 (FIG. 6C) is an OTC image corresponding to a vertical cross-section plane 616 indicated in the image 602. Note that the orientation of the tip 612 is such that the internal reference dimension d manifests itself in each of the en face dimensions X, Y and axial dimension Z. As such, respective pixel-to-size scale parameters for all three (X, Y, Z) dimensions can be determined based on the known internal reference dimension d of the subretinal cannula 610. In various examples, the pixel-to-size scale parameters corresponding to different dimensions may differ from one another or be the same.

In some examples, the three (i.e., X, Y, and Z) pixel-to-size scale parameters determined in the block 412 of the method 400, e.g., as described above in reference to FIGS. 6A-6D, are used to compute a corresponding voxel-to-volume scale parameter. In one example, such voxel-to-volume scale parameter is expressed in the units of voxel-to-μL. Herein, the term “voxel” refers to a three-dimensional counterpart to a pixel. Voxels represent objects of a volumetric image on a regular grid in a 3D space. For example, the approximate and adjusted 3D models computed in the blocks 408 and 410, respectively, can be represented using voxels.

In some examples, operations of the block 412 include the electronic controller 120 computing the volume of the adjusted 3D model in the units of voxels. Such computation may be performed by counting (e.g., via summation) the number of voxels substantially enclosed by the boundary (3D surface) of the adjusted 3D model in the voxel grid. Operations of the block 412 further include the electronic controller 120 applying the voxel-to-volume scale parameter to the volume expressed in the units of voxels, thereby obtaining the estimated value of the object volume in the physical volume units, such as microliters, cubic millimeters, and the like. For example, when the above-described processing is applied to the 3D models 502-512 (see FIGS. 5A-5F), the following corresponding estimated injected-volume values are obtained: (i) 5.5 μL for the injection volume represented by the model 502; (ii) 31.7 μL for the injection volume represented by the model 504; (iii) 60.4 μL for the injection volume represented by the model 506; (iv) 88.1 μL for the injection volume represented by the model 508; (v) 112.8 μL for the injection volume represented by the model 510; and (vi) 134.6 μL for the injection volume represented by the model 512.

In a block 414 of the method 400, the electronic controller 120 is configured to perform or initiate an action responsive to the estimated value of the injected volume determined in the block 414. In various examples, such responsive action may include one or more of the following: displaying the injected-volume value on a display screen; color-coding the displayed value or generating a corresponding encoded visual indicator based on relative proximity of the injected-volume value to the target value; generating a sound signal with one or more characteristics (such as pitch or beeping frequency) thereof depending on the relative proximity of the injected-volume value to the target value; and generating the appropriate control signal 128 for the drug delivery system 130. In various examples, the control signal 128 may cause the drug delivery system 130 to regulate (e.g., increase or decrease) the flow rate with which the corresponding fluid 132 is being delivered to the patient 102 or to stop the fluid delivery, e.g., based on a comparison of the current injected-volume value and the target value. For example, the electronic controller 120 may generate the control signal 128 that causes the drug delivery system 130 to gradually reduce the flow rate as the dynamically updated injected-volume value gets closer to the target value and then fully stop the fluid pump when the injected-volume valued reaches or exceeds the target value.

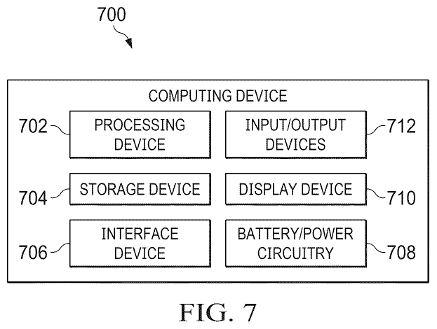

FIG. 7 is a block diagram illustrating a computing device 700 used in or connected to the medical system 100 according to some examples. In various examples, the medical system 100 may include or be communicatively coupled to a single computing device 700 or multiple computing devices 700. In some examples, the computing device 700 implements the electronic controller 120 (also see FIG. 1). In various examples, an instance of the computing device 700 can be used to implement data processing, image processing, video signal generation, and/or one or more system-control functions. In some examples, the computing device 700 is programmed to perform the method 400.

The computing device 700 of FIG. 7 is illustrated as having a number of components, but any one or more of these components may be omitted or duplicated, as suitable for the application and setting. In some embodiments, some or all of the components included in the computing device 700 may be attached to one or more motherboards and enclosed in a housing. In some embodiments, some of those components may be fabricated onto a single system-on-a-chip (SoC) (e.g., the SoC may include one or more electronic processing devices 702 and one or more storage devices 704). Additionally, in various embodiments, the computing device 700 may not include one or more of the components illustrated in FIG. 7, but may include interface circuitry for coupling to the one or more components using any suitable interface (e.g., a Universal Serial Bus (USB) interface, a High-Definition Multimedia Interface (HDMI) interface, a Controller Area Network (CAN) interface, a Serial Peripheral Interface (SPI) interface, an Ethernet interface, a wireless interface, or any other appropriate interface). For example, the computing device 700 may not include a display device 710, but may include display device interface circuitry (e.g., a connector and driver circuitry) to which an external display device 710 may be coupled.

The computing device 700 includes a processing device 702 (e.g., one or more processing devices). As used herein, the terms “electronic processor device” and “processing device” interchangeably refer to any device or portion of a device that processes electronic data from registers and/or memory to transform that electronic data into other electronic data that may be stored in registers and/or memory. In various embodiments, the processing device 702 may include one or more digital signal processors (DSPs), application-specific integrated circuits (ASICs), central processing units (CPUs), graphics processing units (GPUs), server processors, field programmable gate arrays (FPGA), or any other suitable processing devices.

The computing device 700 also includes a storage device 704 (e.g., one or more storage devices). In various embodiments, the storage device 704 may include one or more memory devices, such as random-access memory (RAM) devices (e.g., static RAM (SRAM) devices, magnetic RAM (MRAM) devices, dynamic RAM (DRAM) devices, resistive RAM (RRAM) devices, or conductive-bridging RAM (CBRAM) devices), hard drive-based memory devices, solid-state memory devices, networked drives, cloud drives, or any combination of memory devices. In some embodiments, the storage device 704 may include memory that shares a die with the processing device 702. In such an embodiment, the memory may be used as cache memory and include embedded dynamic random-access memory (eDRAM) or spin transfer torque magnetic random-access memory (STT-MRAM), for example. In some embodiments, the storage device 704 may include non-transitory computer readable media having instructions thereon that, when executed by one or more processing devices (e.g., the processing device 702), cause the computing device 700 to perform any appropriate ones of the methods disclosed herein below or portions of such methods.

The computing device 700 further includes an interface device 706 (e.g., one or more interface devices 706). In various embodiments, the interface device 706 may include one or more communication chips, connectors, and/or other hardware and software to govern communications between the computing device 700 and other computing devices. For example, the interface device 706 may include circuitry for managing wireless communications for the transfer of data to and from the computing device 700. The term “wireless” and its derivatives may be used to describe circuits, devices, systems, methods, techniques, communications channels, etc., that may communicate data via modulated electromagnetic radiation through a nonsolid medium. The term does not imply that the associated devices do not contain any wires, although in some embodiments they might not. Circuitry included in the interface device 706 for managing wireless communications may implement any of a number of wireless standards or protocols, including but not limited to Institute for Electrical and Electronic Engineers (IEEE) standards including Wi-Fi (IEEE 802.11 family), IEEE 802.16 standards, Long-Term Evolution (LTE) project along with any amendments, updates, and/or revisions (e.g., advanced LTE project, ultramobile broadband (UMB) project (also referred to as “3GPP2”), etc.). In some embodiments, circuitry included in the interface device 706 for managing wireless communications may operate in accordance with a Global System for Mobile Communication (GSM), General Packet Radio Service (GPRS), Universal Mobile Telecommunications System (UMTS), High Speed Packet Access (HSPA), Evolved HSPA (E-HSPA), or LTE network. In some embodiments, circuitry included in the interface device 706 for managing wireless communications may operate in accordance with Enhanced Data for GSM Evolution (EDGE), GSM EDGE Radio Access Network (GERAN), Universal Terrestrial Radio Access Network (UTRAN), or Evolved UTRAN (E-UTRAN). In some embodiments, circuitry included in the interface device 706 for managing wireless communications may operate in accordance with Code Division Multiple Access (CDMA), Time Division Multiple Access (TDMA), Digital Enhanced Cordless Telecommunications (DECT), Evolution-Data Optimized (EV-DO), and derivatives thereof, as well as any other wireless protocols that are designated as 3G, 4G, 5G, and beyond. In some embodiments, the interface device 706 may include one or more antennas (e.g., one or more antenna arrays) configured to receive and/or transmit wireless signals.

In some embodiments, the interface device 706 may include circuitry for managing wired communications, such as electrical, optical, or any other suitable communication protocols. For example, the interface device 706 may include circuitry to support communications in accordance with Ethernet technologies. In some embodiments, the interface device 706 may support both wireless and wired communication, and/or may support multiple wired communication protocols and/or multiple wireless communication protocols. For example, a first set of circuitry of the interface device 706 may be dedicated to shorter-range wireless communications such as Wi-Fi or Bluetooth, and a second set of circuitry of the interface device 706 may be dedicated to longer-range wireless communications such as global positioning system (GPS), EDGE, GPRS, CDMA, WiMAX, LTE, EV-DO, or others. In some other embodiments, a first set of circuitry of the interface device 706 may be dedicated to wireless communications, and a second set of circuitry of the interface device 706 may be dedicated to wired communications.

The computing device 700 also includes battery/power circuitry 708. In various embodiments, the battery/power circuitry 708 may include one or more energy storage devices (e.g., batteries or capacitors) and/or circuitry for coupling components of the computing device 700 to an energy source separate from the computing device 700 (e.g., to AC line power).

The computing device 700 also includes a display device 710 (e.g., one or multiple individual display devices). In various embodiments, the display device 710 may include any visual indicators, such as a heads-up display, a computer monitor, a projector, a touchscreen display, a liquid crystal display (LCD), a light-emitting diode display, or a flat panel display.

The computing device 700 also includes additional input/output (I/O) devices 712. In various embodiments, the I/O devices 712 may include one or more data/signal transfer interfaces, audio I/O devices (e.g., microphones or microphone arrays, speakers, headsets, earbuds, alarms, etc.), audio codecs, video codecs, printers, sensors (e.g., thermocouples or other temperature sensors, humidity sensors, pressure sensors, vibration sensors, etc.), image capture devices (e.g., one or more cameras), human interface devices (e.g., keyboards, cursor control devices, such as a mouse, a stylus, a trackball, or a touchpad), etc.

Depending on the specific embodiment of the system 100, various components of the interface devices 706 and/or I/O devices 712 can be configured to send and receive suitable control messages, suitable control/telemetry signals, and streams of data. In some examples, the interface devices 706 and/or I/O devices 712 include one or more analog-to-digital converters (ADCs) for transforming received analog signals into a digital form suitable for operations performed by the processing device 702 and/or the storage device 704. In some additional examples, the interface devices 706 and/or I/O devices 712 include one or more digital-to-analog converters (DACs) for transforming digital signals provided by the processing device 702 and/or the storage device 704 into an analog form suitable for being communicated to the corresponding components of the system 100.

According to an example embodiment disclosed above, e.g., in the summary section and/or in reference to any one or any combination of some or all of FIGS. 1-7, provided is an apparatus comprising: a drug delivery system configurable to controllably inject a fluid into an organ of a patient; an optical instrument configured to perform volumetric imaging in a field of view (FOV) including a surgical instrument and an injection site in the organ; and an electronic controller configured to: determine a pixel-to-size scale parameter based on a pixelated image of the surgical instrument in the FOV; and estimate a volume of the fluid injected by the drug delivery system into the organ based on a pixelated volumetric image of the FOV and further based on the pixel-to-size scale parameter.

Herein, the term “fluid” refers to a material that may continuously move and deform (e.g., flow) under an applied shear stress or external force or pressure gradient. Fluids are substances that cannot significantly resist a shear force applied to them. In medicine and biology, the term “fluid” may also be used to refer to any (quasi) liquid constituent of the body. In some cases, liquids that are given for fluid replacement or medicine delivery, either by drinking or by injection, are also referred to as fluids. Examples of fluids include but are not limited to gases, liquids, solutions, colloids, suspensions, and homogeneous, quasi-homogeneous, or heterogeneous mixtures of several components in a liquid carrier. One example of a fluid is a saline suspension of cells, e.g., suitable for stem-cell therapy.

In some embodiments of the above apparatus, the fluid comprises a drug or a therapeutic substance.

In some embodiments of any of the above apparatus, the organ is an eye.

In some embodiments of any of the above apparatus, the surgical instrument is selected from the group consisting of surgical forceps, a surgical knife or blade, a light pipe, an endo-illuminator, a cannula, a needle, a surgical pick, a surgical brush, and a surgical scraper.

In some embodiments of any of the above apparatus, the electronic controller is further configured to generate a control signal for the drug delivery system based on a difference between the estimated volume of the fluid and a target volume.

In some embodiments of any of the above apparatus, the drug delivery system is configured to regulate a flow rate of the fluid into the organ or to stop the fluid injection in response to the control signal.

In some embodiments of any of the above apparatus, the target volume is smaller than 200 (or 100, or 500, or 1000) microliters.

In some embodiments of any of the above apparatus, the electronic controller is further configured to compute a sequence of estimated volume values, each of the estimated volume values corresponding to a different respective time after a start time of the fluid injection.

In some embodiments of any of the above apparatus, the sequence of estimated volume values is computed in real time.

In some embodiments of any of the above apparatus, the optical instrument is configured to perform volumetric imaging using intraoperative spectrally encoded coherence tomography and reflectometry.

According to another example embodiment disclosed above, e.g., in the summary section and/or in reference to any one or any combination of some or all of FIGS. 1-7, provided is a method of quantifying an injected volume in an organ during a medical procedure, the method comprising: determining a pixel-to-size scale parameter based on a pixelated image of a reference object included in a FOV of an optical instrument configured to perform volumetric imaging, the FOV further including an injection site in the organ; computing a first 3D model of the injected volume in the organ based on a volumetric image of the FOV obtained using the optical instrument; refining the first 3D model to obtain a second 3D model of the injected volume in the organ, the refining including correcting a shape and a size of the first 3D model to reduce distortions associated with light refraction at a boundary of the injected volume in the organ; and calculating a value of the injected volume based on the second 3D model and further based on the pixel-to-size scale parameter.

In some embodiments of the above method, the organ is an eye.

In some embodiments of any of the above methods, the reference object is a surgical instrument selected from the group consisting of surgical forceps, a surgical knife or blade, a light pipe, an endo-illuminator, a cannula, a needle, a surgical pick, a surgical brush, and a surgical scraper.

In some embodiments of any of the above methods, the method further comprises performing or initiating a responsive action in a medical system used to perform the medical procedure, the responsive action being based on the calculated value.

In some embodiments of any of the above methods, the responsive action comprises generating a control signal for a drug delivery system of the medical system based on a difference between the calculated value and a target value.

In some embodiments of any of the above methods, the responsive action comprises generating a control signal for a drug delivery system of the medical system based on a time series of the calculated values, the control signal being configured to change a flow rate with which the drug delivery system delivers the fluid to the organ or area of tissue.

In some embodiments of any of the above methods, the computing comprises: detecting an object representing the injected volume in a plurality of slices of the volumetric image; performing image segmentation within a bounding box corresponding to the detected object to identify respective segments of the object in different ones of the slices; and applying interpolation to a stack of the identified respective segments to obtain a 3D surface representing the boundary of the injected volume in the organ.

In some embodiments of any of the above methods, the refining comprises: optical ray-casting through a top portion of the obtained 3D surface based on Snell's law; correcting a shape of a bottom portion of the obtained 3D surface based on the ray-casting; and scaling the size of the first 3D model based on a refractive index of the injected volume and further based on a refractive index of tissue surrounding the injected volume in the organ.

In some embodiments of any of the above methods, the calculating comprises: calculating a volume of the second 3D model in a voxel grid; determining a voxel-to-volume scale parameter based on the pixel-to-size scale parameter; and calculating the value of the injected volume by applying the voxel-to-volume scale parameter to the calculated volume of the second 3D model.

In some embodiments of any of the above methods, the method further comprises applying one or more preprocessing operations to a sequence of image frames acquired by the optical instrument to generate the volumetric image of the FOV.

Some embodiments provide a non-transitory computer-readable medium storing instructions that, when executed by an electronic processor, cause the electronic processor to perform operations comprising any one of the above methods.

With regard to the processes, systems, methods, heuristics, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating certain embodiments and should in no way be construed so as to limit the claims.

Accordingly, it is to be understood that the above description is intended to be illustrative and not restrictive. Many embodiments and applications other than the examples provided would be apparent upon reading the above description. The scope should be determined, not with reference to the above description, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

All terms used in the claims are intended to be given their broadest reasonable constructions and their ordinary meanings as understood by those knowledgeable in the technologies described herein unless an explicit indication to the contrary is made herein. In particular, use of the singular articles such as “a,” “the,” “said,” etc. should be read to recite one or more of the indicated elements unless a claim recites an explicit limitation to the contrary.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments incorporate more features than are expressly recited in each claim. Rather, as the following claims reflect, inventive subject matter lies in fewer than all features of a single disclosed embodiment. Thus, the following claims are hereby incorporated into the Detailed Description, with each claim standing on its own as a separately claimed subject matter.

While this disclosure includes references to illustrative embodiments, this specification is not intended to be construed in a limiting sense. Various modifications of the described embodiments, as well as other embodiments within the scope of the disclosure, which are apparent to persons skilled in the art to which the disclosure pertains are deemed to lie within the principle and scope of the disclosure, e.g., as expressed in the following claims.

Some embodiments may be implemented as circuit-based processes, including possible implementation on a single integrated circuit.

Some embodiments can be embodied in the form of methods and apparatuses for practicing those methods. Some embodiments can also be embodied in the form of program code recorded in tangible media, such as magnetic recording media, optical recording media, solid state memory, floppy diskettes, CD-ROMs, hard drives, or any other non-transitory machine-readable storage medium, wherein, when the program code is loaded into and executed by a machine, such as a computer, the machine becomes an apparatus for practicing the patented invention(s). Some embodiments can also be embodied in the form of program code, for example, stored in a non-transitory machine-readable storage medium including being loaded into and/or executed by a machine, wherein, when the program code is loaded into and executed by a machine, such as a computer or a processor, the machine becomes an apparatus for practicing the patented invention(s). When implemented on a general-purpose processor, the program code segments combine with the processor to provide a unique device that operates analogously to specific logic circuits.

Unless explicitly stated otherwise, each numerical value and range should be interpreted as being approximate as if the word “about” or “approximately” preceded the value or range.

The use of figure numbers and/or figure reference labels in the claims is intended to identify one or more possible embodiments of the claimed subject matter in order to facilitate the interpretation of the claims. Such use is not to be construed as necessarily limiting the scope of those claims to the embodiments shown in the corresponding figures.

Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

Reference herein to “one embodiment” or “an embodiment” means that a particular feature, structure, or characteristic described in connection with the embodiment can be included in at least one embodiment of the disclosure. The appearances of the phrase “in one embodiment” in various places in the specification are not necessarily all referring to the same embodiment, nor are separate or alternative embodiments necessarily mutually exclusive of other embodiments. The same applies to the term “implementation.”

Unless otherwise specified herein, the use of the ordinal adjectives “first,” “second,” “third,” etc., to refer to an object of a plurality of like objects merely indicates that different instances of such like objects are being referred to, and is not intended to imply that the like objects so referred-to have to be in a corresponding order or sequence, either temporally, spatially, in ranking, or in any other manner.

Unless otherwise specified herein, in addition to its plain meaning, the conjunction “if” may also or alternatively be construed to mean “when” or “upon” or “in response to determining” or “in response to detecting,” which construal may depend on the corresponding specific context. For example, the phrase “if it is determined” or “if [a stated condition] is detected” may be construed to mean “upon determining” or “in response to determining” or “upon detecting [the stated condition or event]” or “in response to detecting [the stated condition or event].”

Also, for purposes of this description, the terms “couple,” “coupling,” “coupled,” “connect,” “connecting,” or “connected” refer to any manner known in the art or later developed in which energy is allowed to be transferred between two or more elements, and the interposition of one or more additional elements is contemplated, although not required. Conversely, the terms “directly coupled,” “directly connected,” etc., imply the absence of such additional elements.

The functions of the various elements shown in the figures, including any functional blocks labeled as “processors” and/or “controllers,” may be provided through the use of dedicated hardware as well as hardware capable of executing software in association with appropriate software. When provided by a processor, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (DSP) hardware, network processor, application specific integrated circuit (ASIC), field programmable gate array (FPGA), read only memory (ROM) for storing software, random access memory (RAM), and nonvolatile storage. Other hardware, conventional and/or custom, may also be included. Similarly, any switches shown in the figures are conceptual only. Their function may be carried out through the operation of program logic, through dedicated logic, through the interaction of program control and dedicated logic, or even manually, the particular technique being selectable by the implementer as more specifically understood from the context.

As used in this application, the terms “circuit,” “circuitry” may refer to one or more or all of the following: (a) hardware-only circuit implementations (such as implementations in only analog and/or digital circuitry); (b) combinations of hardware circuits and software, such as (as applicable): (i) a combination of analog and/or digital hardware circuit(s) with software/firmware and (ii) any portions of hardware processor(s) with software (including digital signal processor(s)), software, and memory (ies) that work together to cause an apparatus, such as a mobile phone or server, to perform various functions); and (c) hardware circuit(s) and or processor(s), such as a microprocessor(s) or a portion of a microprocessor(s), that requires software (e.g., firmware) for operation, but the software may not be present when it is not needed for operation.” This definition of circuitry applies to all uses of this term in this application, including in any claims. As a further example, as used in this application, the term circuitry also covers an implementation of merely a hardware circuit or processor (or multiple processors) or portion of a hardware circuit or processor and its (or their) accompanying software and/or firmware. The term circuitry also covers, for example and if applicable to the particular claim element, a baseband integrated circuit or processor integrated circuit for a mobile device or a similar integrated circuit in server, a cellular network device, or other computing or network device.

It should be appreciated by those of ordinary skill in the art that any block diagrams herein represent conceptual views of illustrative circuitry embodying the principles of the disclosure. Similarly, it will be appreciated that any flow charts, flow diagrams, state transition diagrams, pseudo code, and the like represent various processes which may be substantially represented in computer readable medium and so executed by a computer or processor, whether or not such computer or processor is explicitly shown.

The modifier “about” or “approximately” used in connection with a quantity is inclusive of the stated value and has the meaning dictated by the context (for example, it includes at least the degree of error associated with the measurement of the particular quantity). The modifier “about” or “approximately” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so that, for example, “about 1” may also mean from 0.5 to 1.4.

“SUMMARY” in this specification is intended to introduce some example embodiments, with additional embodiments being described in “DETAILED DESCRIPTION” and/or in reference to one or more drawings. “SUMMARY” is not intended to identify essential elements or features of the claimed subject matter, nor is it intended to limit the scope of the claimed subject matter.

What is claimed is:

1. A medical system, comprising:
a drug delivery system configurable to controllably inject a fluid into an area of tissue of a patient;
an optical instrument configured to perform volumetric imaging in a field of view (FOV) including a surgical instrument and an injection site in the area of tissue; and
an electronic controller configured to:
determine a pixel-to-size scale parameter based on a pixelated image of the surgical instrument in the FOV; and
estimate a volume of the fluid injected by the drug delivery system into the area of tissue based on a pixelated volumetric image of the FOV and further based on the pixel-to-size scale parameter.

2. The medical system of claim 1, wherein the fluid comprises a drug or a therapeutic substance.

3. The medical system of claim 1, wherein the area of tissue is in an eye of the patient.

4. The medical system of claim 1, wherein the surgical instrument is selected from the group consisting of:
surgical forceps,
a surgical knife or blade,
a light pipe,
an endo-illuminator,
a cannula,
a needle,
a surgical pick,
a surgical brush, and
a surgical scraper.

5. The medical system of claim 1, wherein the electronic controller is further configured to generate a control signal for the drug delivery system based on a difference between the estimated volume of the fluid and a target volume.

6. The medical system of claim 5, wherein the drug delivery system is configured to regulate a flow rate of the fluid into the area of tissue or to stop the fluid injection in response to the control signal.

7. The medical system of claim 5, wherein the target volume is smaller than 1000 microliters.

8. The medical system of claim 1, wherein the electronic controller is further configured to compute a sequence of estimated volume values, each of the estimated volume values corresponding to a different respective time after a start time of the fluid injection.

9. The medical system of claim 8, wherein the sequence of estimated volume values is computed in real time.

10. The medical system of claim 1, wherein the optical instrument is configured to perform volumetric imaging using intraoperative spectrally encoded coherence tomography and reflectometry.

11. A method of quantifying an injected volume in an area of tissue during a medical procedure, the method comprising:
determining a pixel-to-size scale parameter based on a pixelated image of a reference object included in a field of view (FOV) of an optical instrument configured to perform volumetric imaging, the FOV further including an injection site in the area of tissue;
computing a first three-dimensional (3D) model of the injected volume in the area of tissue based on a volumetric image of the FOV obtained using the optical instrument;
refining the first 3D model to obtain a second 3D model of the injected volume in the area of tissue, the refining including correcting a shape and a size of the first 3D model to reduce distortions associated with light refraction at a boundary of the injected volume in the area of tissue; and
calculating a value of the injected volume based on the second 3D model and further based on the pixel-to-size scale parameter.

12. The method of claim 11, wherein the area of tissue is in an eye of a patient.

13. The method of claim 11, wherein the reference object is a surgical instrument selected from the group consisting of:
surgical forceps,
a surgical knife or blade,
a light pipe,
an endo-illuminator,
a cannula,
a needle,
a surgical pick,
a surgical brush, and
a surgical scraper.

14. The method of claim 11, further comprising performing or initiating a responsive action in a medical system used to perform the medical procedure, the responsive action being based on the calculated value.

15. The method of claim 14, wherein the responsive action comprises generating a control signal for a drug delivery system of the medical system based on a difference between the calculated value and a target value.

16. The method of claim 14, wherein the responsive action comprises generating a control signal for a drug delivery system of the medical system based on a time series of the calculated values, the control signal being configured to change a flow rate with which the drug delivery system delivers the fluid to the area of tissue.

17. The method of claim 11, wherein the computing comprises:
detecting an object representing the injected volume in a plurality of slices of the volumetric image;
performing image segmentation within a bounding box corresponding to the detected object to identify respective segments of the object in different ones of the slices; and
applying interpolation to a stack of the identified respective segments to obtain a 3D surface representing the boundary of the injected volume in the area of tissue.

18. The method of claim 17, wherein the refining comprises:
optical ray-casting through a top portion of the obtained 3D surface based on Snell's law;
correcting a shape of a bottom portion of the obtained 3D surface based on the ray-casting; and
scaling the size of the first 3D model based on a refractive index of the injected volume and further based on a refractive index of tissue surrounding the injected volume in the area of tissue.

19. The method of claim 18, wherein the calculating comprises:
calculating a volume of the second 3D model in a voxel grid;
determining a voxel-to-volume scale parameter based on the pixel-to-size scale parameter; and
calculating the value of the injected volume by applying the voxel-to-volume scale parameter to the calculated volume of the second 3D model.

20. A non-transitory computer-readable medium storing instructions that, when executed by an electronic processor, cause the electronic processor to perform operations comprising the method of claim 11.
